(19) United States
(12) Patent Application Publication
(10) Pub. No.: US 2015/0159991 A1
(43) Pub. Date: Jun. 11, 2015

(54) OCT DEVICE

(71) Applicant: Sumitomo Electric Industries, Ltd., Osaka-shi (JP)

(72) Inventor: Takemi HASEGAWA, Yokohama-shi (JP)

(21) Appl. No.: 14/562,084

(22) Filed: Dec. 5, 2014

(30) Foreign Application Priority Data
Dec. 6, 2013 (JP) 2013-253289

(51) Int. Cl. G01B 9/02; A61B 5/00

(52) U.S. Cl. CPC G01B 9/02083; G01B 9/02091; G01B 9/02049; A61B 5/0066; A61B 5/0084

(57) ABSTRACT

Provided is an OCT device capable of restraining degradation of OCT image quality and cost increase of parts and generating a scan trigger signal. The OCT device of the present invention comprises: a light source for outputting laser light having oscillation wavelength and output power both changing with time; an interference optical system, in which first laser light output from the light source is divided, into measurement light and reference light such that the measurement light is output so as to irradiate the object and the reference light is caused to pass through a reference light path, and the measurement light back-scattered from the object and the reference light having passed through the reference light path are combined to be output, the transmissivity of the interference optical system having a wavelength dependence opposite to the wavelength dependence of the first output power of the light source; and an OCT photodetector for generating an OCT electrical signal with a value according to interference light intensity by detecting interference light which is a combination of the reference light and the measurement light outputted from the interference optical system.

### FIELD OF THE INVENTION

The present invention relates to an OCT device for obtain tomographic image of an object by using optical interference.

### BACKGROUND ART

The optical coherence tomography (OCT) is known as a technology for obtaining a tomographic image of living tissues such as a blood vessel. According to the OCT technology, the distribution of optical reflectance on the optical path of measurement light is measured by detecting and analyzing the interference light which is a combination of the light reflected from an object irradiated with measurement light and the light which has passed through the reference light path.

It is possible to achieve two-dimensional or three dimensional measurement for the distribution of optical reflectance in a section of a blood vessel by performing OCT measurement using a catheter containing an optical fiber such that measurement light is irradiated to a blood vessel wall from the lumen of the blood vessel and by scanning the intravascular wall with the measurement light. The plaque composition can be discriminated on the basis of a blood vessel tomographic image obtained by OCT, since the intima, media and adventitia which constitute a blood vessel, as well as the lipid, calcic, and fibrous substance which constitute a plaque lesion, have respectively different reflective characteristics.

Swept Source Optical Coherence Tomography (SS-OCT) is known as a method for realizing OCT. The SS-OCT uses a wavelength swept light source in which a laser oscillation wavelength changes periodically as a function of time. The output light from the wavelength swept light source is separated into measurement light and reference light, and the interference light formed as a result of interference between the measurement light which has been irradiated to an object and reflected by the object, and the reference light which has passed through the reference light path are detected and converted into an electric signal by an optical detector. And, the spectrum of interference light can be acquired by recording the electric signal as a function of time, and the distribution of the optical reflectance on the optical path of measurement light by conducting frequency analysis of the spectrum.

In SS-OCT, an electric signal obtained as a function of time must be converted into the function of wave number (equivalent to the reciprocal of wavelength), and therefore it is necessary to measure the relation between time and the oscillation wavelength of a light source. In particular, it is necessary to convert the electric signal as a function of time into the function of a wavelength on the basis of a scan trigger signal generated by detecting a timing at which the wavelength sweep reaches a given phase.

According to a known method for generating a scan trigger signal, the timing at which an oscillation wavelength reaches a specific wavelength is found by detecting the output light of a light source through Fiber Bragg Grating (FBG) Which reflects light of specific wavelength (Japanese Patent Application Laid-Open No. 2012-239514). As another known method, the timing at which the power of output light from a light source reaches a given threshold value is detected by monitoring the output light power. The latter method uses the fact that the change in a wavelength of light output from a wavelength swept light source accompanies change in the output power of the light.

### SUMMARY OF THE INVENTION

### Object of the Invention

An object of the present invention is to provide an OCT device which is capable of generating a scan trigger signal and which is suitable fur restraining degradation in quality of OCT image and increase in cost of parts.

### Means for Achieving the Object

An OCT device of the present invention for obtaining a tomographic image of an object by using interference of light comprises: (1) a light source for outputting first laser light and second laser light, the first laser light having an oscillation wavelength and a first output power both changing with time, and the second laser light having a second output power substantially proportional to the first output power; (2) interference optical system in which the first laser fight is divided into measurement light and reference light such that the measurement light is outputted so as to irradiate the object and the reference light is caused to pass through a reference light path, and the measurement light back-scattered from the object and the reference light having passed through the reference light path are combined to be output, the transmissivity of the interference optical system having wavelength dependence that is opposite to the wavelength dependence of the first output power of the light source; (3) an OCT photodetector for generating an OCT electrical signal with a value according to interference light intensity by detecting the interference light, which is a combination of the reference light and the measurement light outputted from the interference optical system; (4) a monitor photodetector for generating a first monitor electrical signal according to the second output power by detecting the second laser light; (5) an AD converter for changing the OCT electrical signal into a digital signal and outputting the digital signal during the period set according to the first monitor electrical signal; and (6) an analysis unit for generating optical coherence tomographic image data by conducting frequency an of the digital signal.

### Advantageous Effect of the Invention

According to the present invention, a scan trigger signal can be generated and the degradation of quality of an OCT image and the increase in cost of parts can be restrained.

### BRIEF DESCRIPTION OF DRAWINGS

FIG. 1 is a schematic diagram of an OCT device according to the first embodiment of the present invention.

FIG. 2 is a schematic diagram showing a section of OCT catheter in the distal end of the OCT device of FIG. 1.

FIG. 3 shows characteristics of the OCT device of FIG. 1.

FIG. 4 shows characteristics of the OCT device of FIG. 1.

FIG. 5 shows characteristics of the OCT device of a comparative example.

FIG. 6 shows characteristics of the OCT device of a comparative example.

FIG. 7 is a schematic diagram of an OCT device according to the second embodiment of the present invention.

FIG. 8 shows characteristics of the OCT device of FIG. 7.

### DETAILED DESCRIPTION OF THE INVENTION

The method of detecting a timing by using FBG has a problem in which an error occurs as the reflective wavelength of FBG changes according to temperature or distortion. Another problem of the method is increase in cost of equipment parts because of FBG. The method of detecting the timing by monitoring the output power of a light source does not have problems due to the FRG and has simple composition. In this case, in order to achieve highly accurate detection of the timing at which the threshold value is reached, it is desirable that the change in power accompanying the wavelength change of a light source be large. However, if the change of power is enlarged, the power of interference light also significantly changes according to the wavelength, which will result in the problem in which the quality of OCT image is degraded because SN ratio decreases at some wavelengths, or a detector is saturated.

Hereafter, with reference to accompanying drawings, examples for carrying out the present invention will be described in detail. In the explanation of the drawings, the same mark will be given to identical elements, and overlapping explanation mill be omitted. The present invention is not limited to the examples and is shown by claims, including all modifications equivalent to or within the scope of a claim.

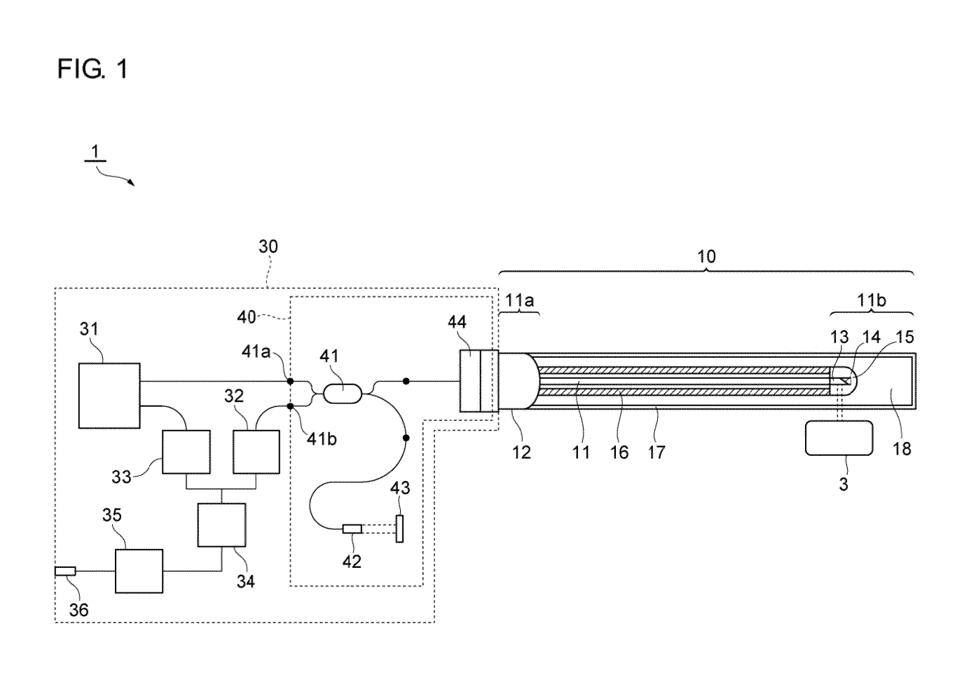

FIG. 1 is a schema to diagram of OCT device 1 according to the first embodiment of the present invention. OCT device 1 is equipped with OCT catheter 10 and measurement unit 30 and obtains an OCT image of object 3 by photographing.

The OCT catheter 10 has an optical fiber 11, an optical connector 12, a focusing optical system 13 and a deflecting optical system 14, a cap 15, a support tube 16, and a jacket tube 17. The optical fiber 11 transmits light between the proximal end 11a and the distal end 11b. The optical connector 12 is connected with the optical fiber 11 at the proximal end 11a. The focusing optical system 13 and the deflecting optical system 14 are optically connected with the optical fiber 11 at the distal end 11b. The cap 15 surrounds the focusing optical system 13 and the deflecting optical system 14. The support tube 16 and the jacket tube 17 extend, surrounding the optical fiber 11. The lumen of the jacket tribe 17 is filled with medium 18. The optical connector 12 is optically and electrically connected to a probe-rotation and transfer mechanism 44 which is a part of the measurement unit 30.

The optical fiber 11 is made of silica glass and has a length of 1 m to 3 m. The optical fiber 11 has an attenuation of 2 dB or less, preferably 1 dB or less, in the wavelength range of 1.2 μm to 1.4 μm, or 1.6 μm to 1.8 μm and operates in a single mode in the above-mentioned wavelength range. As for such optical fiber 11, an optical fiber based on ITU-T G.652, G.654, and 6.657 is suitable.

A graded index (GRIN) lens as the focusing optical system 13 is fusion-spliced to the distal end 11b of the optical fiber 11. The tip of the GRIN lens has an inclined end face reflecting light and functions as the deflecting optical system 14. The light is emitted converging in the radial direction by passing through the focusing optical system 13 and the deflecting optical system 14.

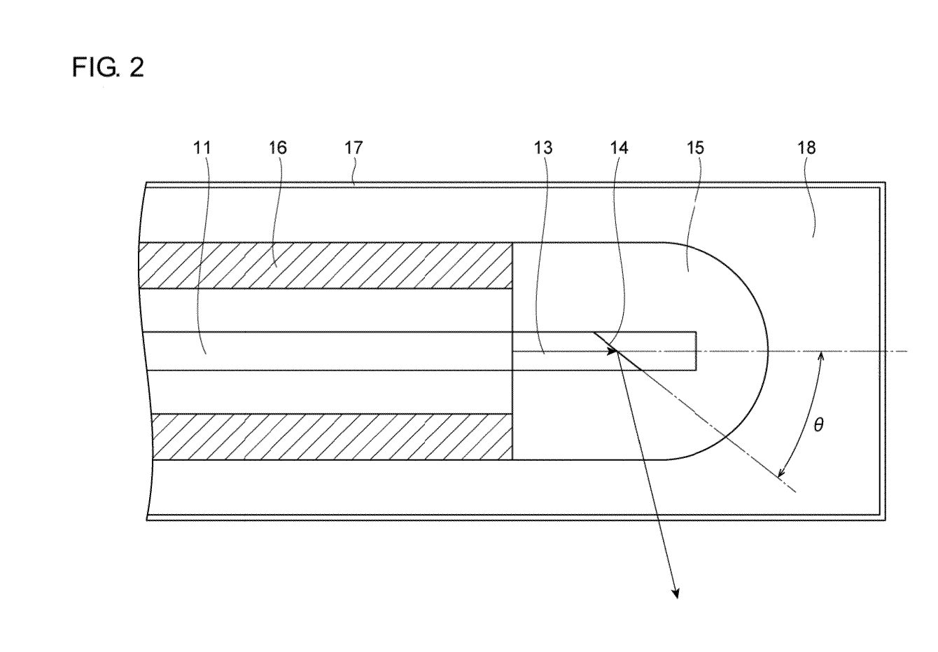

The GRIN lens (focusing optical system 13 and deflecting optical system 14) is made of silica glass or borosilicate glass and has an attenuation of 2 dB or less in a wavelength range of 1.2 μm to 1.4 μm, or a wavelength range of 1.6 μm to 1.8 μm. As shown in FIG. 2, a flat reflective surface which acts as a mirror is formed on an end of columnar glass at an angle θ of 35 degree to 44 degree relative to the axis of the GRIN lens. The flat reflective surface can reflect light as it is. In order to enhance reflectance, however, it is preferable to coat the reflective surface with vapor deposition of aluminum or gold.

The cap 15 made of urethane acrylate resin or epoxy resin and has an attenuation of 2 dB or less in the wavelength range of 1.2 μm to 1.4 μm, or 1.6 μm to 1.8 μm. The cap 15 mechanically protects the focusing optical system 13 and the deflecting optical system 14 and has a function to realize a total reflection mirror by confining air so as to touch the interface of the mirror of the deflecting optical system 14.

The optical fiber 11 is stored in the lumen of the support tube 16, which is fixed to the optical connector 12 and the tip portion of the optical fiber 11. As a result, when the optical connector 12 is rotated, the support tube 16 also rotates together, and the rotational torque is transmitted to the optical fiber 11, so that the optical fiber 11, the focusing optical system 13, the deflecting optical system 14, the cap 15, and the support tube 16 rotate altogether. Therefore, the torque applied to the optical fiber 11 is reduced as compared with the case where only the optical fiber 11 is rotated, and consequently the fracture of optical fiber 11 which might otherwise be caused by torque can be prevented.

It is desirable that the support tube 16 have not only a thickness of 0.15 mm or more, but also Young's modulus of 100 GPa to 300 GPa, which is equivalent to stainless steel. The support tube 16 may not necessarily be connected in the circumferential direction, and may have a structure in which about 5 to 20 strands of wires are twisted together, so that the flexibility may be adjusted.

The optical fiber 11, the focusing optical system 13, the deflecting optical system 14, the cap 15, and the support tube 16 are stored in the lumen of the jacket tube 17, where they can be rotated. Therefore, the object 3 can be prevented from being damaged in the case where a rotating part contacts the object 3. The illumination light is emitted from the deflecting optical system 14, penetrates the cap 15, the medium 18, and the jacket tube 17, and is irradiated to the object 3.

The jacket tube 17 is made of any of polyamide (e.g., nylon, polyether block amide), fluoro-resin (e.g., FEP, PFA, PTFE), polyester (PET), and polyolefin (e.g., polyethylene, polypropylene), has a thickness of μm 30 to 100 μm, and has such a transparency as the transmission loss is 2 dB or less at an wavelength of 1.2 μm to 1.4 μm, or 1.6 μm to 1.8 μm. When the thickness of the jacket tube 17 is thicker than the spatial resolution of OCT measurement, which is usually 30 μm or less, the reflections of inner and external surfaces of the jacket tube 17 can be used for calibration, such as dispersion compensation, if the reflections are detected by making distinction among them.

The medium 18 filled in the lumen of the jacket tube 17 is liquid or gas. The medium 18 is chosen so that it may have a refractive index different from the jacket tube 17. Consequently, light is partially reflected at the interface between the jacket tube 17 and the medium 18. The light partially reflected is detected by the measurement unit 30 in the same manner as light back-scattered at the object 3. Therefore, the partially reflected light can be used in order to confirm whether the optical fiber 11 transmits the light. The materials suitable for the medium 18 are silicone oil, physiological saline, dextran solution, air, nitrogen gas, carbon dioxide, etc., because they are suitable for the above-mentioned purpose.

The measurement unit 30 has light source 31, interference optical system 40, OCT photodetector 32, monitor photodetector 33, AD converter 34, analysis unit 35, and output port 36. The light source 31 outputs, from two ports, laser light, the oscillation wavelength and the output power of which change with time. The interference optical system 40 divide the first laser light outputted from the first port of the light source 31 into measurement light and reference light, and outputs it after transmission. The OCT photodetector 32 outputs an OCT electrical signal by detecting interference light which is a combination of measurement light an reference light. The monitor photodetector 33 detects the second laser light outputted from the second port of the light source 31, and outputs a laser light power monitor electrical signal (first monitor electrical signal). The AD converter 34 outputs a digital signal by digitizing the required section of OCT electrical signal on the basis of the first monitor electrical signal. The analysis unit 35 performs signal processing, such as frequency analysis, for digital signals, generates tomographic images of the object 3, and outputs the image data of the tomographic images. The output port 36 outputs the image data to the exterior for display or record.

The two output ports of the light source 31 may be configured by branching a part of light in a laser resonator from each of two separate parts in the resonator. The two output ports of the light source 31 may be configured by further dividing the light branched from one common part of the resonator. The light source 31 and the monitor photodetector 33 may be housed in one box as a light source system. In such case, the light source system outputs the first laser light and the first monitor electrical signal. Although the first monitor electrical signal outputted may be an analog signal corresponding to the light power of the second laser light, it may be a digital signal which is output when the analog signal reaches at a threshold value, whereby the influence of noise can be reduced.

The interference optical system 40 has an optical coupler 41, a probe-rotation and transfer mechanism 44, a reference port 42, and a reference minor 43. The optical coupler 41 inputs into the first port 41a the light outputted from the first port of the light source 31, divides the input light into measurement light and reference light and outputs the measurement light and the reference light from separate ports. The probe-rotation and transfer mechanism 44 combines the measurement light with the optical fiber 11 in the optical connector 12 of the OCT catheter 10. The reference port 42 outputs reference light to the space in the equipment. The reference mirror 43 allows the outputted reference light to pass through a predetermined optical path length, and thereafter couples it to a reference port again by reflection.

The measurement light outputted from the probe-rotation and transfer mechanism 44 is connected to the proximal end 11a of the optical fiber 11 by the optical connector 12 and guided by the optical fiber 11 so as to be emitted from the distal end 11b, and the light thus emitted is irradiated to the object 3 through the focusing optical system 13, the deflecting optical system 14, and the cap 15. The measurement light back-scattered at the object 3 enters into the distal end 11b of the optical fiber 11 through the cap 15, the deflecting optical system 14, and the focusing optical system 13, so that it is guided by the optical fiber 11 so as to be outputted from the proximal end 11a. The output light is connected to the probe-rotation and transfer mechanism 44 by the optical connector 12, passes through the optical coupler 41 so as to be outputted from the second port 41b of the optical coupler 41, and enters into the OCT photodetector 32. The reference light reflected at the reference mirror 43 is connected to the reference port 42 so as to pass through the optical coupler 41, so that it is outputted from the second port 41b of the optical coupler 41, and enters the OCT photodetector 32. The interference light which is a combination of the measurement light hack-scattered at the object 3 and the reference light is detected by the OCT photodetector 32, and whereby an OCT electrical signal having a value depending on the power of the interference light is generated in the OCT photodetector 32.

The monitor photodetector 33 detects the light outputted from the second port of the light source 31, and the first monitor electrical signal having a value depending on the power of the light is generated in the monitor photodetector 33. The OCT electrical signal generated in the OCT photodetector 32 and the first monitor electrical signal generated in the monitor photodetector 33 are input into the analysis unit 35. The spectrum of interference light is analyzed by the analysis unit 35, and distribution of the optical back-scattering efficiency in each point inside the object 3 is calculated. On the basis of the calculation results, the tomographic image of the object 3 is calculated as OCT image signal and outputted from the output port 36.

In the first embodiment, the transmissivity of the interference optical system 40 has wavelength dependence which is opposite to that of the output power of the light source 31. Here, the transmissivity of the interference optical system 40 is the transmissivity of measurement light, or the transmissivity of reference light. At least either the transmissivity of reference light or the transmissivity of the measurement light in the interference optical system 40 has the wavelength dependence that is opposite to the wave lob dependence of the output power of the light source 31. The transmissivity of measurement light is a product of the transmissivity from the first port 41a of the optical coupler 41 to the probe-rotation and transfer mechanism 44 multiplied by the transmissivity from the probe-rotation and transfer mechanism 44 to the second port 41b of the optical coupler 41. The transmissivity of reference light is the transmissivity of an optical path from the first port 41a of the optical coupler 41 to the second port 41b of the optical coupler 41 via the reference mirror 43. The term “wavelength dependence is opposite” does not necessarily require that the opposite relationship be strictly reciprocal, but it is acceptable if the sign of slope of wavelength dependence is mutually opposite when linear approximation is carried out in the oscillation wavelength range of the light source 31.

In the first embodiment, measurement light and reference light enter into an OCT photodetector, and an electric signal is proportionally generated in accordance with the sum of measurement light power, reference light power, and the power of interference light which is a combination of the measurement light and the reference light. In this case, it is desirable to make the reference light power sufficiently larger than the measurement light power, whereby the noise caused in the OCT image by multiple reflection in the measurement optical path can be reduced. Therefore, it is desirable that the transmissivity of reference light have wavelength dependence which is opposite to the wavelength dependence of the output power of the light source 31.

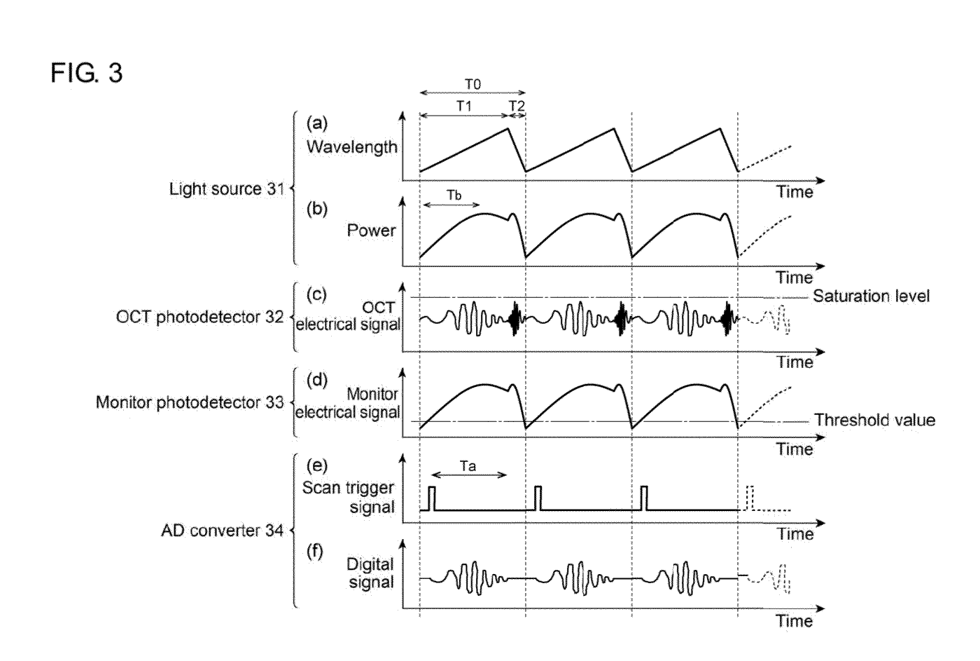

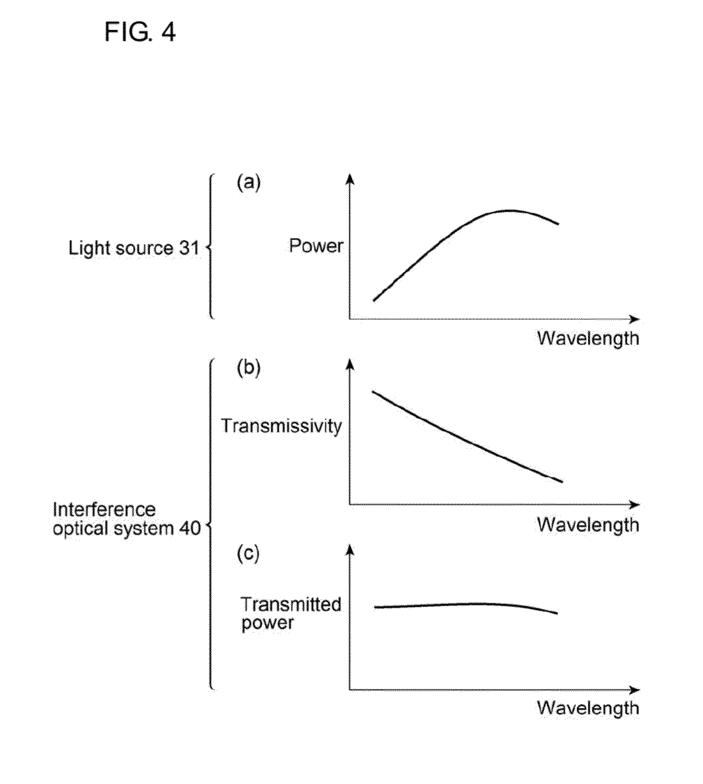

FIGS. 3 and 4 illustrate the characteristics of OCT device 1 of the first embodiment. FIG. 3 shows the respective temporal variation with respect to: (a) the oscillation wavelength of light source 31; (b) the output power of light source 31; (c) OCT electrical signal output from OCT photodetector 32; (d) first monitor electrical signal output from monitor photodetector 33; (e) scan trigger signal in AD converter 34; and (f) digital signal output from AD converter 34. FIG. 4 shows the respective wavelength dependence with respect to: (a) the output power of light source 31; (b) the transmissivity of interference optical system 40; and (c) the transmitted power of interference optical system 40.

As shown m FIG. 3 (a), the oscillation wavelength of the light source 31 is swept periodically as a function of time in a sawtooth shape. The wavelength sweep cycle is T0, and in each cycle T0, the period T1 is a sweep in the direction for longer wavelength, and the period T2 is a sweep in the direction for shorter wavelength. Since the characteristics of the light sources 31, such as coherence length and noise, may change depending on the sweep direction, it is suitable for OCT measurement to use only the sweep in the direction for longer wavelength (or only the sweep in the direction for shorter wavelength). However, in the present invention it is also possible to conduct the sweep in both directions. The output power of the light source 31 has wavelength dependence as shown in FIG. 4 (a). Therefore, as shown in FIG. 3 (b), the output power of the light source 31 also changes periodically as a function of time along with the sweep of a wavelength.

In the first embodiment, as shown in (a) and (b) of FIG. 4, the transmissivity of the interference optical system 40 has wavelength dependence opposite to the wavelength dependence of the output power of the light source 31. Since the interference optical system 40 has such a transmission spectrum, the wavelength dependence of the transmitted power of the interference optical system 40 is reduced as shown in (c) of FIG. 4. The transmitted power of the interference optical system 40 is a product of the output power of the light source 31 and the transmissivity of the interference optical system 40.

In the case of reference light, the transmitted light power calculated from the transmissivity of the reference light in the interference optical system 41 is equal to the reference light power in the second port 41b of the optical coupler 41. On the other hand, in the case of measurement light, since the transmissivity of OCT catheter 10 and the transmissivity of the object 3 are not contained, the transmitted light power calculated from the transmissivity of the measurement light in the interference optical system 41 is not necessarily in agreement with the measurement light power in the second port 41b of the optical coupler 41. However, since the transmissivity of OCT catheter 10 and the transmissivity of the object 3 may be changed at random in the case of the latter, the wavelength dependence of the measurement light power in the second port 41b of the optical coupler 41 is reduced statistically. By reducing the wavelength dependence of the transmitted power of the interference optical system 40, as shown in FIG. 4 (c), as shown in FIG. 3 (c), a temporal change in which the OCT electrical signal outputted from OCT photodetector originates in change of the output power of the light source 31 is controlled.

On the other hand, synchronizing with the sweep of a wavelength, the output power of the light source 31 changes periodically, and it increases in monotone in accordance with time over time Tb from the start of the sweep to the long wavelength direction. Thereby, as shown in FIG. 3 (d), the first monitor electrical signal outputted from the monitor photodetector 33 changes periodically as a function of time, and it increases in monotone to time over the time Tb in a similar manner.

In AD converter 34, when the first monitor electrical signal changes beyond a predetermined threshold value in the predetermined direction, a trigger signal for directing commencement of conversion (scan trigger signal) is generated as shown at (e) of FIG. 3 and when the trigger signal for directing commencement of conversion occurs, the OCT electrical signal is recorded by converting from analog to digital over the retrieve time Ta. The threshold value so as to fall within the variation range of the first monitor electrical signal in the time Ta. The retrieve time Ta is set as a time obtained by subtracting the predetermined margin from the sweep time T1. As a result, a digital signal is obtained as shown at (f) of FIG. 3. An OCT image data is obtained by conducting frequency analysis of the digital signal thus obtained. Instead of acquiring a digital signal over the predetermined retrieve time Ta, digital values may be acquired by predetermined retrieving sample number N, or a trigger signal for directing the finish of conversion may be generated by setting another threshold value in the first monitor electrical signal.

The output power of light source 31 can be changed by synchronizing with the sweep of a wavelength. However, because of a factor different from that, the output power of the light source 31 may gradually change with environmental temperature etc. Such change will cause an error in the timing of the scanning trigger generated by the above-mentioned method. In order to reduce such error, it is desirable that the light source 31 be controlled so that the average value of the output power in a time longer than the cycle of wavelength sweep may become constant. Such control can be achieved, for example, by adjusting the gain inside at light source on the basis of difference if a measurement value is different from a predetermined value when the output power of the light source is measured.

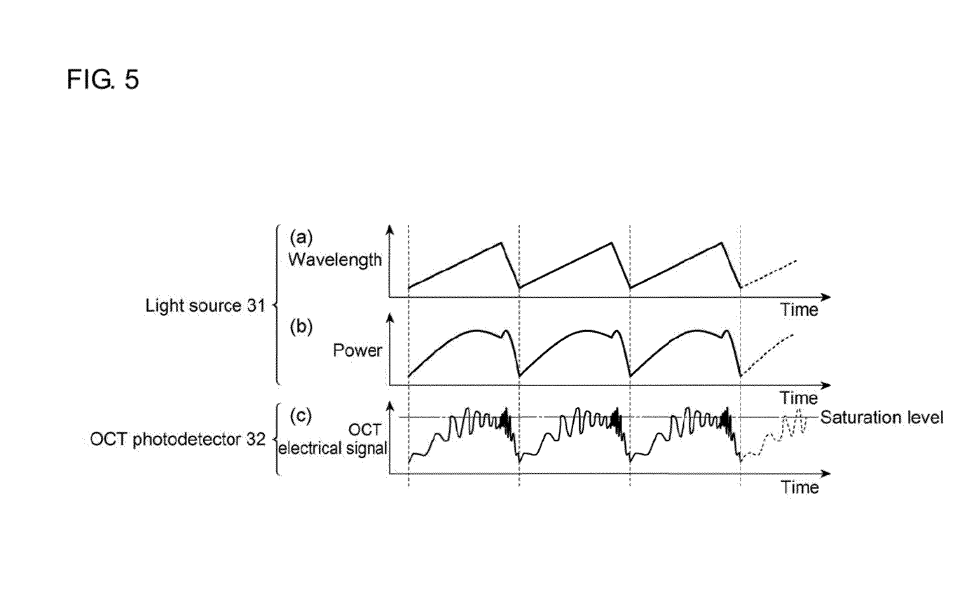

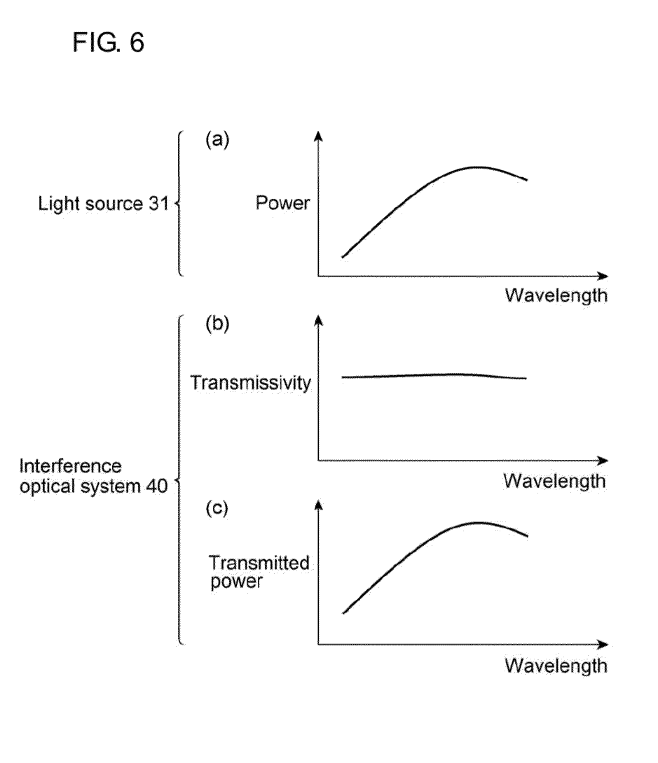

Here, a comparative example to be contrasted with the first embodiment will be explained. FIGS. 5 and 6 show characteristics of the OCT device of a comparative example. FIG. 5 shows the respective temporal change with respect to: (a) oscillation wavelength of light source 31; (b) output power of light source 31; and (c) OCT electrical signal output from OCT photodetector 32. FIG. 6 shows the respective wavelength dependence with respect to: (a) output power of light source 31; (b) transmissivity of interference optical system 40; and (e) transmitted power of interference optical system 40.

As shown at (a) of FIG. 5, the oscillation wavelength of the light source 31 is swept with time, and as shown at (a) of FIG. 6, the output power of the light source 31 has wavelength dependence. Therefore, as shown in FIG. 5 (b), the output power of the light source 31 also changes with time. However, in the comparative example, as shown at (a) and (b) of FIG. 6, the transmissivity of the interference optical system 40 does not have wavelength dependence that is opposite to the wavelength dependence of the output power of the light source 31, and the transmissivity is substantially constant without depending on the wavelength. Therefore, as shown in FIG. 6 (c), the transmitted power of the interference optical system 40 significantly changes with time. As a result, as shown in FIG. 5 (c), the OCT electrical signal output from the OCT photodetector 32 changes sharply with time.

Consequently, in the comparative example, the value of the OCT electrical signal will exceed the saturation level of the AD converter 34 in some periods. The OCT electrical signal exceeding the saturation level is not correctly converted from analog to digital by the AD converter 34, and in such case, the OCT image data generated from the digital signal will have degraded image quality. It is possible to avoid saturation by attenuating the OCT electrical signal. In that case, however, a low-level portion of the OCT electrical signal will be further attenuated, and the signal to noise ratio will decrease, whereby the image quality of the OCT image data will also be degraded.

In contrast with such problem existing in the comparative example, in the first embodiment the timing of a sweep can be acquired on the basis of temporal change of the output power of the light source 31, and the temporal change of the OCT electrical signal can be controlled. In the first embodiment, it is possible to control the degradation of OCT image quality and increase in cost of parts, and to generate a scan trigger signal. In the first embodiment, it is also possible to prevent degradation of the image quality of OCT image data due to the saturation of the OCT photodetector 32, or the degradation of the signal to noise ratio.

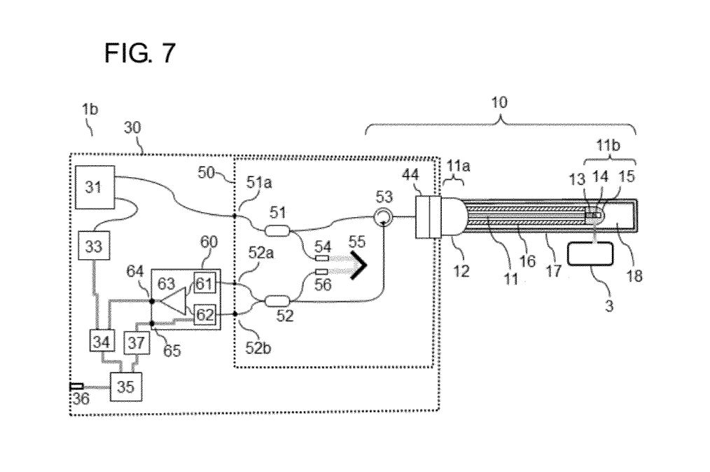

FIG. 7 is a schematic diagram of OCT device 1b of the second embodiment of the present invention. The OCT device 1b differs from the OCT device 1 in that a balanced-type interference optical system 50 is used instead of the interference optical system 40 and a balanced type OCT photodetector 60 is used instead of the OCT photodetector 32.

The interference optical system 50 has is first optical coupler 51 and a circulator 53. The first optical coupler 51 lets the light outputted from the first port of the light source 31 to enter into the first port 51a, divides the light into measurement light and reference light, and outputs the measurement light and the reference light from separate ports respectively. The circulator 53 connects the measurement light to the probe-rotation and transfer mechanism 44, and connects the measurement light from the probe-rotation and transfer mechanism 44 to a second optical coupler 52. The reference light is emitted to a space from the first reference port 54 and connected to the second reference port 56 after passing through the reference light path by a predetermined light path length and turning back at the reference mirror 55. The reference light which has been connected to the second reference port 56 is connected to the second optical coupler 52. In the second optical coupler, the measurement light and the reference light are combined and output from a first output port 52a and a second output port 52b. The measurement light and reference light which are output from the first and second output ports are detected by the balanced-type OCT photodetector 60.

In the balanced-type OCT photodetector 60, the measurement light and reference light which are output from the first output port are detected by a first optical detector 61, and the measurement light and the reference light which are output from the second output port are detected by a second optical detector 62. And in the difference circuit 63, an OCT electrical signal, which is generated by taking and amplifying the difference of an electric signal detected with the two optical detectors 61 and 62, are output from a first electric output port 64. The OCT electrical signal thus outputted is converted from analog to digital by the AD converter 34. From the optical detector 62, an interference light power monitor electrical signal (second monitor electrical signal) corresponding to the power of the output light from an interference optical system is o generated and outputted from a second electric output port 65. The second monitor electrical signal is converted from analog to digital by a second AD converter 37 and is analyzed by the analysis unit 35.

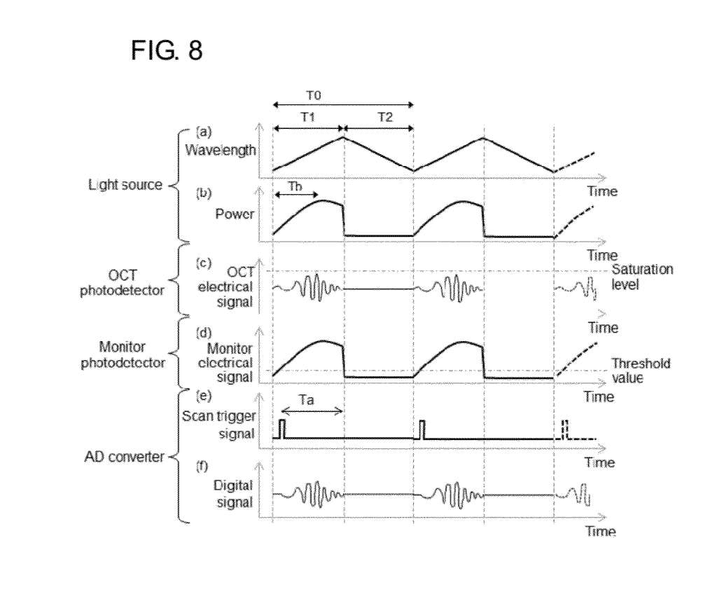

FIG. 8 shows characteristics of the OCT device 1b of the second embodiment. FIG. 8 shows the respective temporal change with respect to: (a) oscillation wavelength of the light source 31; (b) output power of the light source 31; (c) OCT electrical signal output from the balanced-type OCT photodetector 60; (d) first monitor electrical signal output from the monitor photodetector 33; (e) scan trigger signal in the AD converter 34; and (f) digital signal output from the AD converter 34. The output power of the light source 31, the transmissivity of the interference optical system 40, and the transmitted power of the interference optical system 40, their respective wavelength dependence are the same as those shown in FIG. 4.

As shown at (a) of FIG. 8, the sweep of the oscillation wavelength of the light source 31 is periodically carried out as a function of time in a shape of triangular wave. The cycle of a wavelength sweep is T0, and at each cycle T0, a period T1 is a sweep in the direction for longer wavelength, while a period T2 is a sweep in the direction for shorter wavelength. The performance of the light sources 31, such as coherence length and noise, may change depending on the sweep direction, only the sweep in the longer wavelength direction (or only the sweep in the shorter wavelength direction) is output from the light source and used for OCT measurement and the sweep in the shorter wavelength direction is not output. The output power of the light source 31 has wavelength dependence as shown at (a) of FIG. 4. Therefore, as shown at (b) of FIG. 8, the output power of the light source 31 also changes periodically as a function of time along with the wavelength sweep.

In the second embodiment, the amplitude of an OCT electrical signal is proportional to the product of the amplitude of reference light and measurement light, and therefore the transmissivity denoted by the geometric mean of the transmissivity of reference light and the transmissivity of the measurement light of the interference optical system 40 preferably has wav length dependence which is opposite to the wavelength dependence of the output power of the light source 31 as shown at (a) and (b) of FIG. 4. In such case, the wavelength dependence of the transmitted power expressed as a geometric mean between the measurement light and the reference light outputted from the interference optical system 40 is reduced as shown at (c) of FIG. 4. Therefore, as shown art (c) of FIG. 8, the temporal change due to the change of the output power of the light source 31 can be restrained with respect to the OCT electrical signal output from the balanced-type OCT-photodetector 60.

As a result, a first monitor electrical signal, a trigger signal for directing commencement of conversion (scan trigger signal), and a digital signal can be obtained in the same manner as shown at (d) to (f) in FIG. 3. In the second embodiment, the balance detection of measurement light and reference light enables reducing the influence by change of measurement light power and reference light power on the change of the OCT electrical signal, and therefore it is possible to reduce disorder of the OCT image due to saturation of an OCT electrical signal, and accordingly it is possible to obtain an OCT image which has no disorder even in the case of an object in which the reflectance significantly changes.

In order to reduce scan-triggering timing error due to gradual change in the output power of the light source 31, it is preferable that the light source 31 be controlled so that the average value of the output power during a time which is longer than the cycle of a wavelength sweep may become constant. On the other hand, there is a case where the central value of the oscillation wavelength which is periodically swept also gradually changes because of environmental temperature, etc. Such change of main wavelength may cause an analysis error in the case where an analysis is performed for detecting a substance in an object by analyzing the spectrum of the interference light which is a combination of measurement light and reference light.

In the second embodiment, the second monitor electrical signal corresponding to the power of light output from the interference optical system is converted from analog to digital by the second AD converter 37 and analyzed by the analysis unit 35. In the analysis unit 35, an average value is computed by equalizing the second monitor electrical signal during a time which is longer than the cycle of wavelength sweep in the light source 31. The average value represents the average per value of power of the reference light and measurement light output from the interior nee optical system. In the second embodiment, a change in the central value of the oscillation wavelength of the light source can be found by detecting a change in the average value of the second monitor electrical signal, since the output power from the light source is controlled so that the average value may become constant and furthermore, as shown at (b) of FIG. 4, the transmissivity of measurement light and reference light exhibits a monotonic change with respect to the wavelength. Even in the case where the central value of the oscillation wavelength of the light source changes, it is possible to maintain the accuracy of spectral analysis by conducting spectral analysis on the basis of detected change in the wavelength.

What is claimed is:

1. An OCT device for obtaining a tomographic image of an object by using interference of light, comprising:
a light source for outputting first laser light and second laser light, the first laser light having an oscillation wavelength and a first output power, both changing with time, and the second laser light having a second output power substantially proportional to the first output power;
an interference optical system dividing the first laser light into measurement light and reference light such that the measurement light is outputted so as to irradiate the object and the reference light is caused to pass through a reference light path, and combining the measurement light back-scattered from the object and the reference light having passed through the reference light path to be output, the transmissivity of the interference optical system having a wavelength dependence opposite to the wavelength dependence of the first output power of the light source;
an OCT photodetector for generating an OCT electrical signal with a value according to interference light intensity by detecting interference light, the interference light being a combination of the reference light and the measurement light outputted from the interference optical system;
a monitor photodetector for generating a first monitor electrical signal according to the second output power by detecting the second laser light;
an AD converter for changing the OCT electrical signal into a digital signal and outputting the digital signal during the period set according to the first monitor electrical signal; and
an analysis unit for generating optical coherence tomographic image data by conducting frequency analysis of the digital signal.

2. An OCT device according to claim 1, wherein
the laser oscillation wavelength and the output power output from the light source exhibit monotonic gradient change with time for at least a part of time range within a period during which laser light is output;
the output power exhibits monotonic change with a first sign with respect to the wavelength in the wavelength range output for the part of time range, while the transmissivity of the interference optical system exhibits monotonic change with a sign opposite to the first sign with respect to the wavelength in the wavelength range; and
the AD converter decides timing for commencement conversion into a digital signal with respect to the OCT electrical signal by detecting the intensity of the second laser light reaching a threshold in the part of time range.

3. An OCT device according to claim 1, further comprising
an output power monitoring detector for detecting the intensity of light combined with the reference light and the measurement light both output from the interference optical system, wherein
the analysis unit detects change of central value of the laser oscillation wavelength by detecting change of the output power.
